(19) United States
(12) Patent Application Publication
(10) Pub. No.: US 2011/0060188 A1
(43) Pub. Date: Mar. 10, 2011

(54) LOW COST DISPOSABLE MEDICAL FORCEPS TO ENABLE A HOLLOW CENTRAL CHANNEL FOR VARIOUS FUNCTIONALITIES

(75) Inventors: Andre Sharon, Newton, MA (US); Satish Singh, Sharon, MA (US); Irving Bigio, Chestnut Hill, MA (US); Svava Atladottir, Mountain View, CA (US); Douglas Foss, Holliston, MA (US); Patrik Vogtel, Aachen (DE)

(73) Assignees: TRUSTEES OF BOSTON UNIVERSITY, Boston, MA (US); BOSTON MEDICAL CENTER CORPORATION, Boston, MA (US); FRAUNHOFER USA, INC., Plymouth, MI (US)

(21) Appl. No.: 12/920,561

(22) PCT Filed: Mar. 6, 2009
(86) PCT No.: PCT/US09/36360

§ 371 Date: Nov. 23, 2010

Related U.S. Application Data
(60) Provisional application No. 61/034,245, filed on Mar. 6, 2008.

(51) Int. Cl. A61B 1/00; A61B 17/94; A61B 10/02

(52) U.S. Cl. 600/106; 600/564

(57) ABSTRACT

The present invention relates to an endoscopic biopsy forceps (10) with an open central channel. The forceps include a sheath (20) having a proximal end and a distal end, a housing (50) connected with the distal end of the outer sheath, an open channel actuator control means (30) having a proximal end and a distal end and passing through the sheath, an operating means attached to the proximal end of the open channel actuator means, an open channel actuator (60.) attached to a distal end of the open channel actuator control means and having a first projection and second projection, a first jaw (40) having an actuator engagement projection and having a first position and a second position, and a second jaw having an actuator engagement projection and a first position and a second position. The first jaw and second jaw are movably connected to the housing, and when the open channel actuator is moved longitudinally along a body of the instrument, the first jaw moves between the open position and the closed position.

### CROSS REFERENCE TO RELATED APPLICATION

This application claims benefit under 35 U.S.C. §119(e) of the U.S. Provisional Application No. 61/034,245 filed Mar. 6, 2008, the contents of which are incorporated herein by reference in its entirety.

### FIELD OF THE INVENTION

The present invention relates to an endoscopic biopsy forceps with an open central channel that may be used as biopsy forceps, graspers, etc. with the additional capability of use with a central optical fiber or fiber bundle for concurrent optical measurements, such as elastic scattering spectroscopy, fluorescence spectroscopy, Raman spectroscopy, fluorescent microscopy, confocal microscopy, etc. The hollow open central channel is large enough to allow for further functionalities, including additional tooling such as a water port for cleaning, snare for cauterization, spike for collection of multiple biopsy samples or stabilization, vacuum, etc; while maintaining enough volume in the forceps cavity to obtain sufficient tissue sampling for subsequent pathological analysis.

### BACKGROUND OF THE INVENTION

A variety of endoscopic biopsy forceps, graspers, and other related apparatuses have been developed to take samples of tissue or grasp and remove material during endoscopic procedures. Normally, the forceps, which are adapted to cut and remove body tissue for examination, are inserted together with an endoscope deep into a body cavity being examined. The forceps conventionally used in such procedures utilize complex arrangements of linkage assemblies or cam type devices for articulating the jaws of the forceps. As such instruments are of small size, such complexity results in complex machining and manufacturing procedures which greatly increase the cost of such instruments. The multiple connections also increase the amount of play, which may increase the distortion of the movement of the jaws of the device. Thus, present biopsy devices are generally very expensive and, the jaw actuating mechanisms are complex and may be inaccurate.

The small size and number of the linkages and hinge pins also decrease the durability of the biopsy forceps and increase their vulnerably to breakage. This is an important consideration, especially when working within a patient where retrieval of a dissociated part may be difficult or dangerous to the patient. Large numbers of small linkages and hinges also increase the cost and difficulty of manufacturing and assembly.

In conventional biopsy forceps, the intended multiple use of the instrument requires extensive cleaning and sterilizing procedures to be performed to comply with medical standards and use of the instruments. When used multiple times, a biopsy instrument must be sterilized between uses by immersing the contaminated instrument in a suitable chemical sterilizing solution, subjecting the apparatus to sterilization in an autoclave, or some other sterilization procedure. The sterilization and cleaning procedures will often decrease the performance or useful life span of the instrument, thereby magnifying the problem created by the complexity of manufacture and many parts which quickly wear. Further, some devices which are intended only for single use still incorporate complex linkage or cam type devices for proper movement of the biopsy jaws. This greatly inhibits their use as the costs associated with such instruments are normally still very high.

### SUMMARY OF THE INVENTION

The present invention provides a biopsy apparatus, in the form of forceps, graspers or other similar devices, for taking a tissue sample having one or two moving sections. The instabilities created by the multiple links and linkage assemblies of the prior art is reduced by elimination of many of the linkages and particularly the hinge pin as a separate member.

Many of the prior art devices also require that additional space be available surrounding the rigid housing during operation of the forceps to allow for the multiple linkages to move beyond the boundary of the rigid housing or sleeve. The present invention operates completely within the housing. The only portion of the device which moves out beyond its initial perimeter is the jaws as they open to obtain a sample.

In keeping with the foregoing discussion, the present invention takes the form of a jawed endoscopic instrument which has one or two moving jaws. The jaws are pivotally attached to a housing and actuated by open channel actuator attached to an open channel actuator control or mechanism. The actuator and actuator control mechanism moves back and forth along the body of the instrument. In one configuration, different diameter sections of the of the open channel actuator means. The engagement projections engage actuator engagement projections that are part of the base of the jaw. As the actuation mechanism is moved toward the distal end of the instrument, the jaws are moved toward an open position. As the actuation mechanism is moved toward the proximal end of the instrument, the jaws are moved toward a closed position. Optionally, the jaws maybe configured to open to a predetermined, maximum angle. In one embodiment the device is a single use device.

One aspect of the present invention is directed toward an endoscopic biopsy forceps with an open central channel. The forceps include a sheath having a proximal end and a distal end, a housing connected with the distal end of the outer sheath, an open channel actuator control having a proximal end and a distal end and passing through the sheath, an operating means attached to the proximal end of the open channel actuator, an open channel actuator attached to a distal end of the open channel actuator control and having a first projection and second projection, a first jaw having an actuator engagement projection and having a first position and a second position, and a second jaw having an actuator engagement projection and a first position and a second position. The first jaw and second jaw are movably connected to the housing, and when the open channel actuator is moved longitudinally along a body of the instrument, the first jaw moves between the open position and the closed position.

### BRIEF DESCRIPTION OF THE DRAWINGS

FIGS. 1A-1H are schematic drawings showing an embodiment of the jaw.

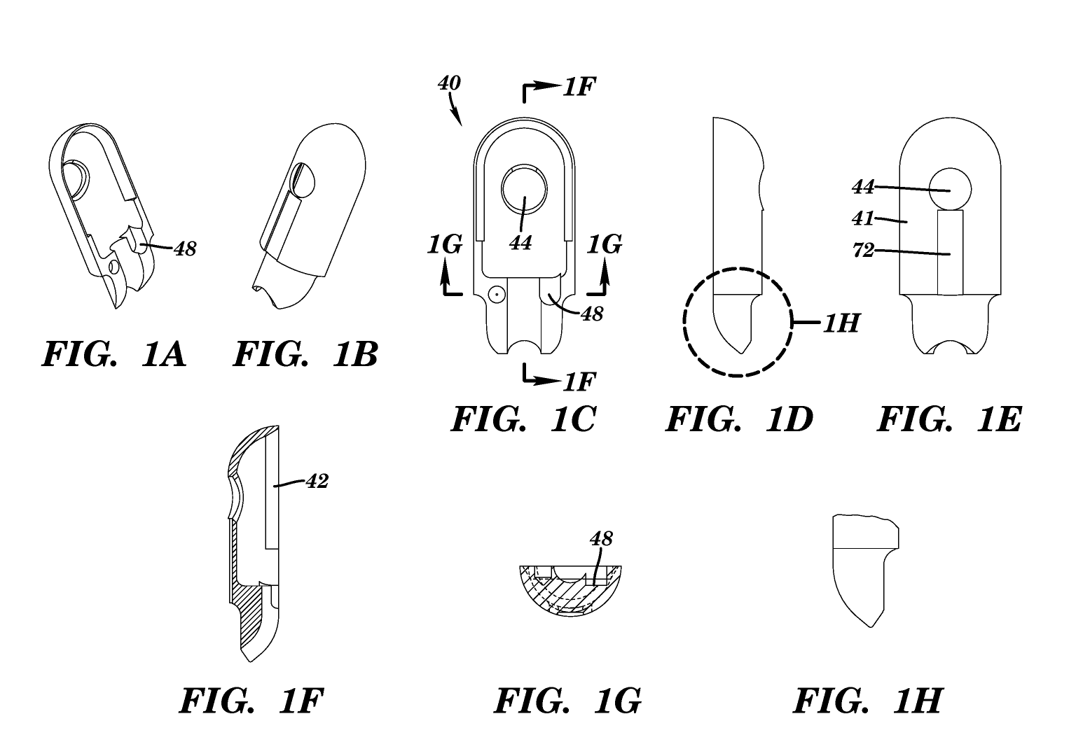

FIGS. 2A-2C are schematic drawings showing an embodiment of the actuator.

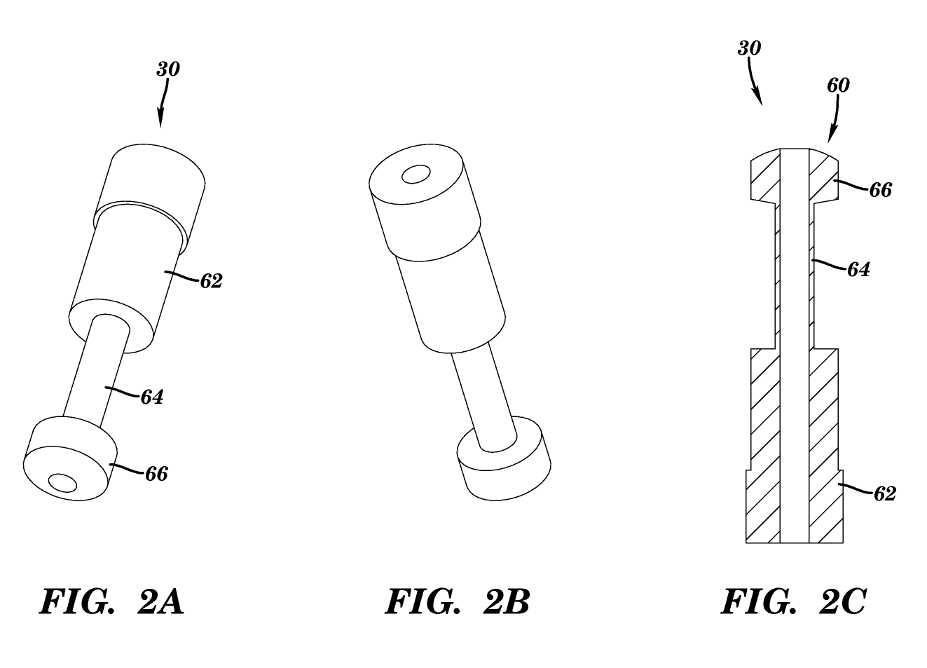

FIGS. 3A-3E are schematic drawings showing an embodiment of the housing.

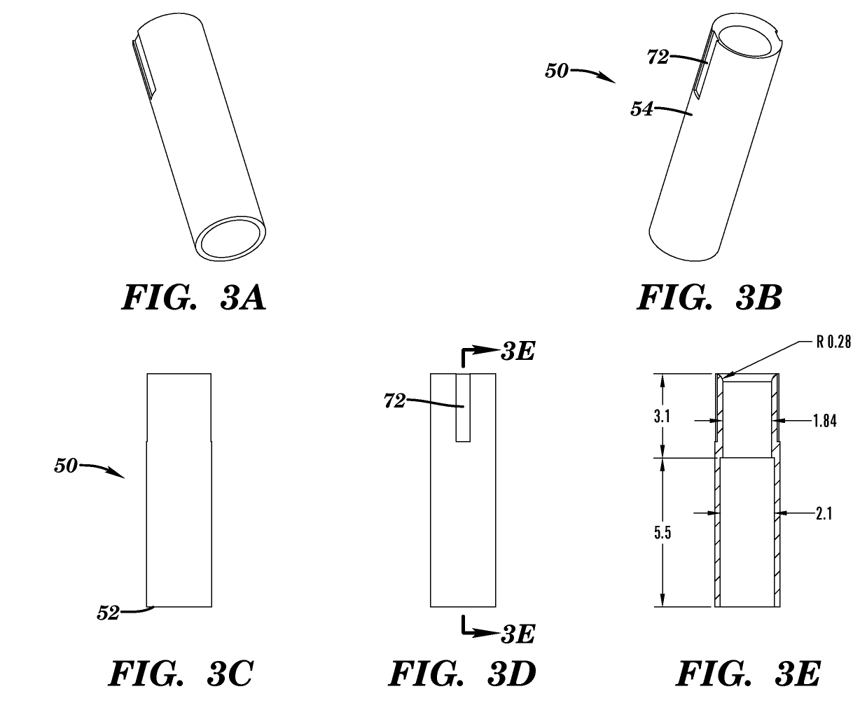

FIGS. 4A-FJ are schematic drawings showing an embodiment of the forceps assembly with ferrule design for forceps jaw opening and closing via camming action.

FIGS. 5A-5B are schematic drawings showing an embodiment of the actuator.

FIGS. 6A-6D are schematic drawings showing an embodiment of the forceps assembly in open and closed positions showing side hinge with flexible joint.

FIG. 7A-D are schematic drawings showing an embodiment of the forceps with ferrule geometry to open and close side hinges. Side view (A) and top view (C) of forceps in open position. Side view (B) of forceps in closed position and top view (0) of the forceps in a partially closed configuration. Note: the length and diameter of the forceps jaws can vary to provide additional volume for tissue or to adjust the force applied in the closed position.

FIGS. 8A-8B are schematic drawings showing an embodiment of the forceps assembly with jaw actuation via balloon. Left) Balloon is fully inflated and the jaws are closed. Right) Balloon is less inflated/deflated and the jaws open.

FIG. 9 is a schematic drawing showing an embodiment of the forceps assembly which can include an ESS fiber with 45 degree angle.

FIGS. 10A-10B are schematic drawings showing an embodiment of the forceps assembly.

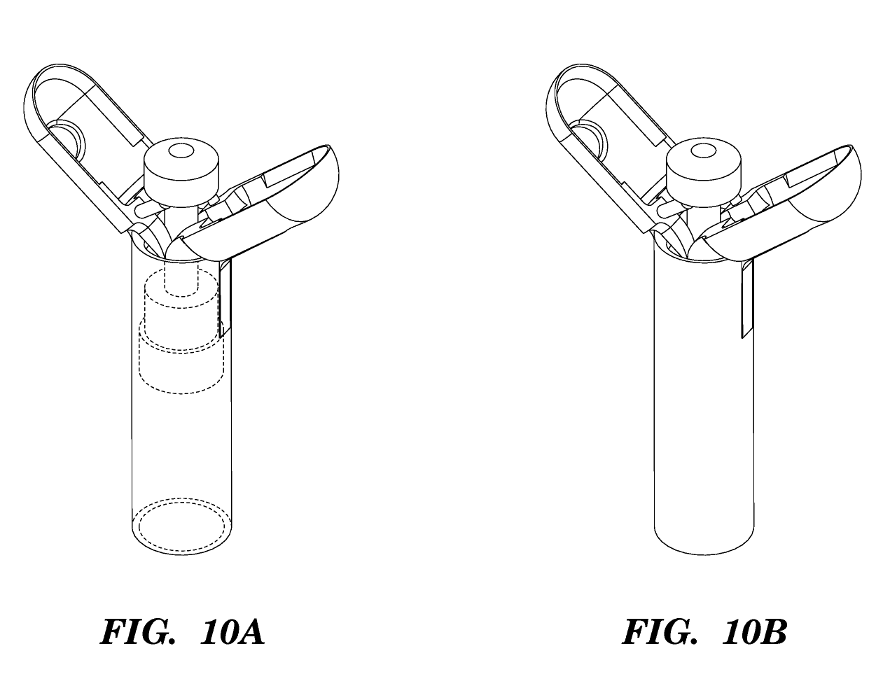

FIGS. 11A-11B are schematic drawings showing an embodiment of the forceps assembly front view before closing.

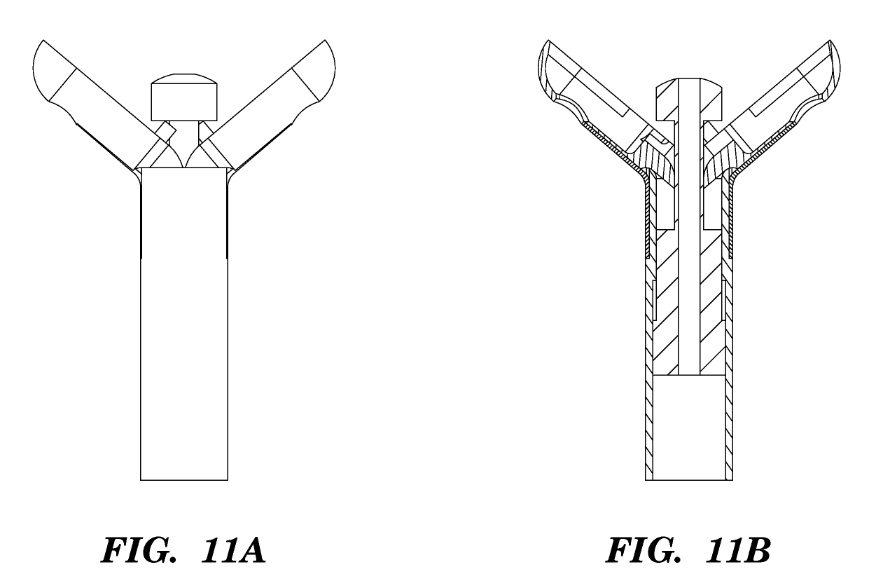

FIGS. 12A-12B are schematic drawings showing an embodiment of the forceps assembly top view before closing.

FIGS. 13A-13B are schematic drawings showing an embodiment of the forceps assembly front view open.

FIGS. 14A-14C are schematic drawings showing an embodiment of the forceps assembly side view open.

FIGS. 15A-15B are schematic drawings showing an embodiment of the forceps assembly closed.

FIGS. 16A-16E are schematic drawings showing an embodiment of the housing.

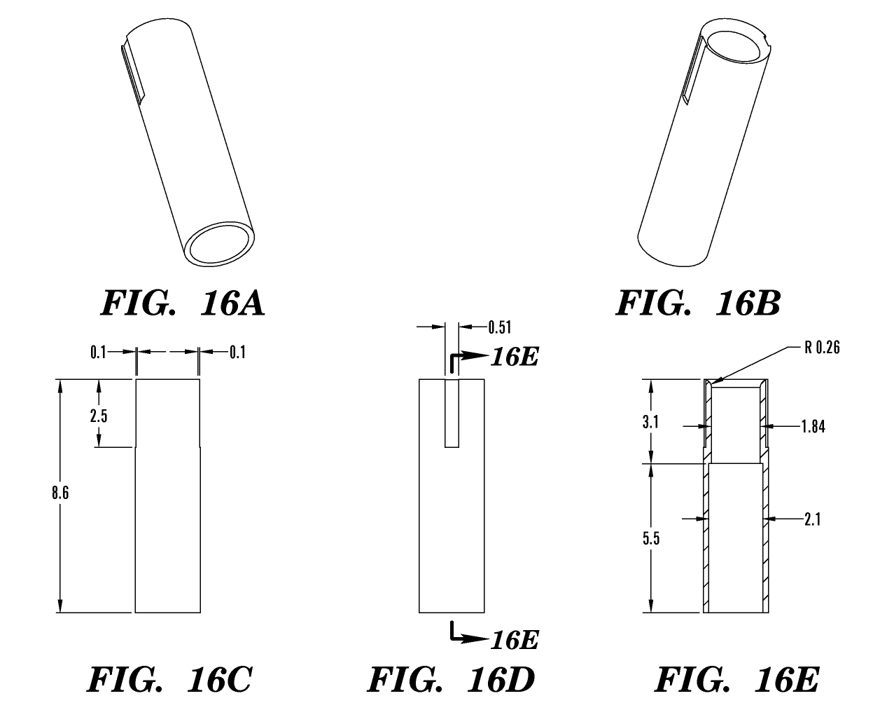

FIGS. 17A-17C are schematic drawings showing an embodiment of the actuator.

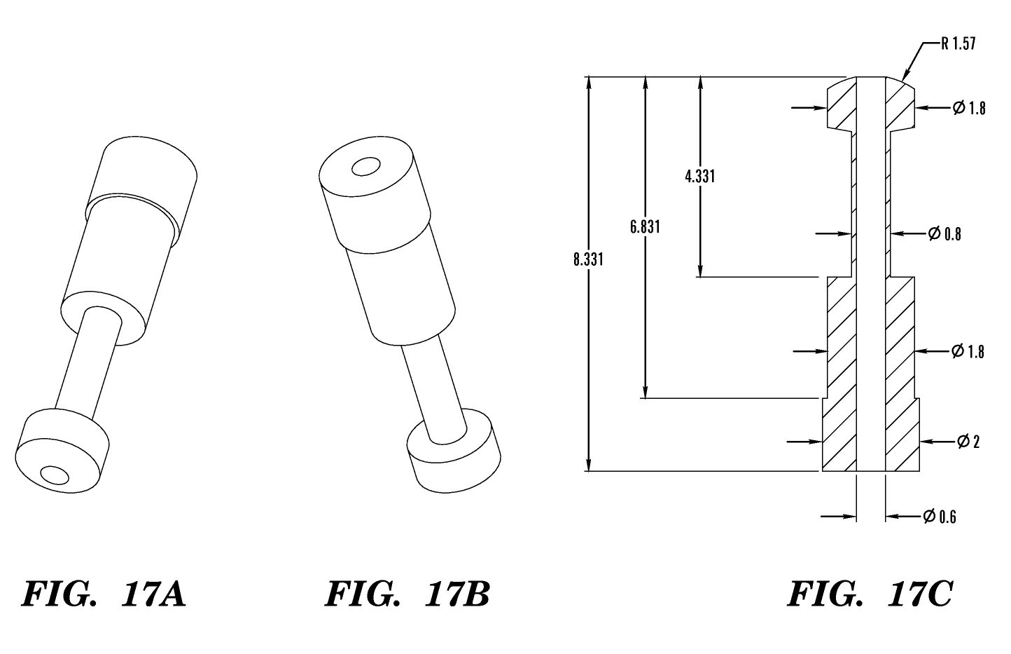

FIGS. 18A-18H are schematic drawings showing an embodiment of the jaw.

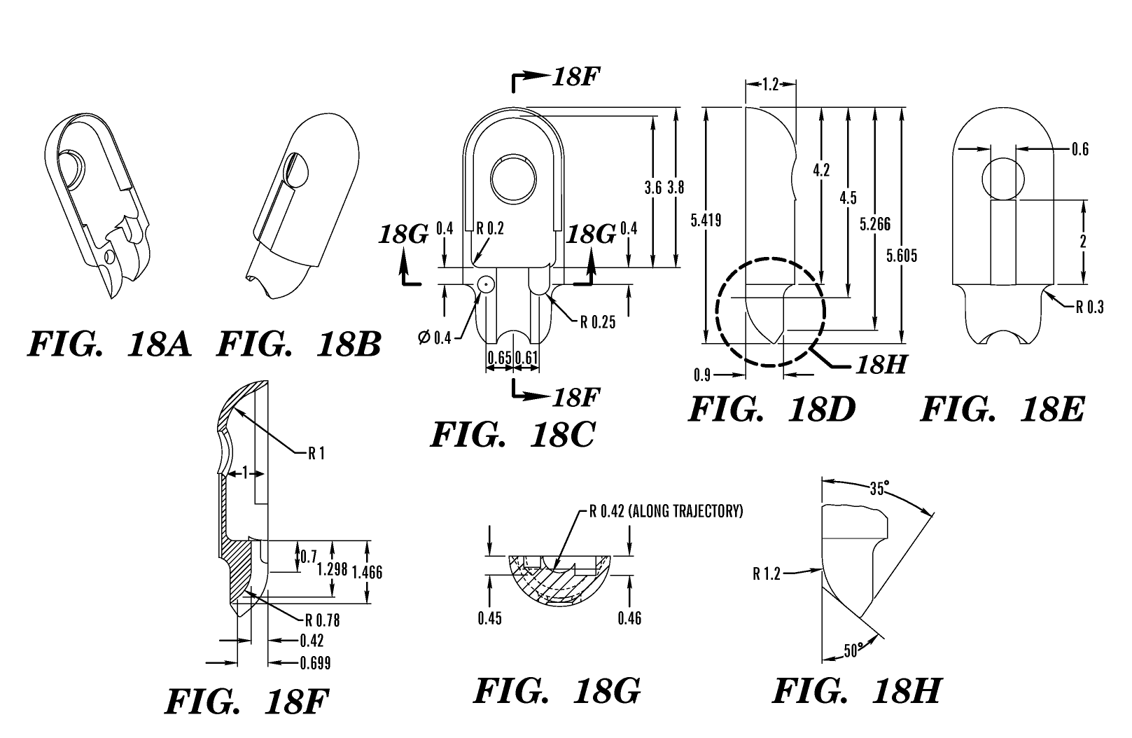

FIGS. 19A-19B are schematic drawings showing an alternate embodiment of the jaw showing forceps concept with shape memory alloys. Left) open position. Right) closed position.

FIG. 20 is a schematic drawing showing an embodiment of the forceps assembly with variation for scanning mucosa.

FIG. 21 is a force description of hinge action. Here, ‘a’ is the length of the jaws, ‘b’ is the diameter, Fclose is the force exerted when the jaws are closed and Fpull is the force exerted to close the jaws.

FIGS. 22A-22B are a schematic describing the relationship between the jaw length and diameter to the maximum force that can be applied during closing. Left) Top view cross-section. Right) Side view cross-section at designated line “A” in right-hand figure. Here, ‘a’ is the length of the jaws and ‘b’ is the diameter.

### DETAILED DESCRIPTION

One aspect of the present invention is directed toward an endoscopic biopsy forceps with an open central channel. The forceps include a sheath having a proximal end and a distal end, a housing connected with the distal end of the outer sheath, an open channel actuator control having a proximal end and a distal end and passing through the sheath, an operator attached to the proximal end of the open channel actuator, an open channel actuator attached to a distal end of the open channel actuator control and having a first projection and second projection, a first jaw having an actuator engagement projection and having a first position and a second position, and a second jaw having an actuator engagement projection and a first position and a second position. The first jaw and second jaw are movably connected to the housing, and when the open channel actuator is moved longitudinally along a body of the instrument, the first jaw moves between the open position and the closed position.

In certain embodiments, the forceps includes a hole extending through at least one of the first jaw and the second jaw. In certain embodiments, the forceps includes jaws that are pivotally connected to the housing by a flexible hinging means. In certain embodiments, the forceps includes jaws that have a cutting edge extending along an upper portion of the jaws.

Although the present invention may take other forms such as graspers, in one embodiment graspers are present. The device shown in the Figures have biopsy forceps 10 for use in endoscopy to, for example, inspect, treat, or take tissue specimens from the body. The biopsy forceps 10 includes a flexible sheath 20, such as a flexible polymeric tubing, coiled steel or the like, having a first end from which control of the forceps 10 is effected by the user. A suitable operating mechanism for actuating the forceps is provided at the first end of the sheath 20 which is connected to open channel actuator control 30. The open channel actuator control 30 is longitudinally movable within the sheath 20, and the suitable operating mechanism will control movement of the open channel actuator control 30 therethrough. The device further includes a pair of biopsy jaws 40 connected to a housing 50 which is fixedly or removably attached to the second end of the sheath 20. The biopsy jaws 40 are operatively connected to housing 50 by a flexible hinge 70, which will be more fully described herein. At least one of the jaws 40 is moveable between open and closed positions with respect to the other of the jaws 40. However, in the embodiment shown, both jaws 40 are moveable between open and closed positions.

The open channel actuator control 30 is preferably an open channel multi-strand cable, but may also be solid, coiled, etc., depending on the requirements or qualities desired provided it defines an central open channel through which further functionalities can be achieved as described in more detail below.

The length of the forceps 10 will vary greatly depending on the intended use. Standard forceps 10 are currently designed in the range of 20-260 centimeters. However, the present invention may be longer or shorter than this range if desired.

The biopsy device 10 shown in the figures has two generally cup-shaped jaws 40. One or both of the jaws 40 preferably has a perimeter which tapers to form a cutting edge 42. If only one of the jaws 40 is moveable and only one has a cutting edge 42, the cutting edge 42 would optimally be located on the moving jaw 40. When moved into the closed position, the jaws 40 cut through the tissue and meet to remove a tissue sample from an organ and contain the sample during the removal process. An optional hole 44 may extend through the wall of one or both of the jaws 40. The hole 44 allows fluid or other extraneous material to escape the jaws 40 as the jaws 40 close, thereby causing less trauma to the sample being removed from the patient. The base 46 of each moveable jaw 40 is configured to allow for clearance and free movement of open channel actuator 60 when jaw 40 is in an open position.

Jaw 40 is operably connected to housing 50 by a flexible hinge 70. The housing 50 is preferably a generally cylindrical body through which the open channel actuator control 30 joins to the open channel actuator 60. The base 52 of the housing 50 is connected to the second end of the sheath 20. The connection may be created by soldering, adhesive, crimping, threading, welding or other known connection methods. Flexible hinge 70 is preferably a strip of flexible material generally rectangular in shape. Flexible hinge 70 may be metal, alloy, plastic, or other suitable material known in the art and may be fastened to housing 50 and jaw 40 by welding, soldering, crimping, adhesives, or other suitable connecting method. Flexible hinge 70 may be a contiguous extension of either housing 50 or jaw 40. Flexible hinge 70 is preferably fitted into flexible hinging means channel 72 in jaw 40 or housing 50 as shown in the figures such that flexible hinging means 70 is substantially flush with housing 50 outer surface 54 and jaw 40 outer surface 41.

Jaw 40 as shown in the figures, is generally cup-shaped. The opposing side of the jaw 40 is preferably shaped the same. Base 46 of jaw 40 is configured to fit within housing 50 and to pivotally move between closed and open positions while allowing free movement of actuator 60 at all times. Base 46 has actuator engagement projection 48.

In the particular configuration shown, movement of the jaws 40 is created by an open channel actuator 60 which is directly connected to the open channel actuator control 30. A generally cylindrical open channel actuator base portion 62 is fixedly or removably attached to the open channel actuator control means 30. The connection may be created by soldering, adhesive, crimping, welding or other known connection methods. Extending from the top of the open channel actuator base portion 62 is a connecting stem 64.

Open channel actuator 60 is operably connected to actuator control means 30 at base portion 62. Said connection may be permanent (e.g. by welding, soldering, or adhesive) or removable (e.g. threaded, Luer, or quick connect fittings). As shown, in one configuration, open channel actuator 60 is substantially cylindrical and progresses from base portion 62 to connecting stem 64. Connecting stem 64 is substantially cylindrical and narrower in diameter than base portion 62 which progresses to actuator tip 66. The diameter of actuator tip 66 is greater than the diameter of connecting stem 64 and may be substantially similar to the diameter of base portion 62. The distal end of actuator tip 66 defines the egress of the open channel defined by open channel actuator 60 and actuator control 30. Actuator tip 66 may be concave, convex, tapered, flat, or any shape suitable for the application desired.

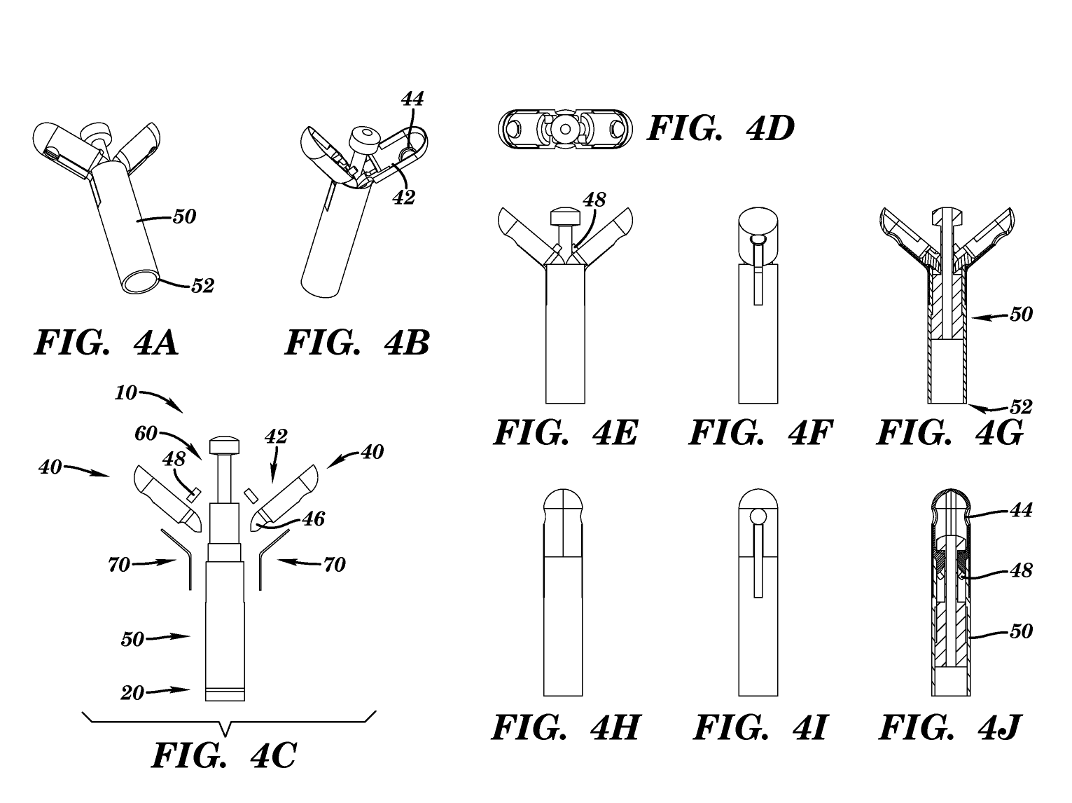

In one configuration, as shown in FIG. 4, when jaw 40 is in a closed position, pushing outward or extension of actuator control 30 causes outward movement of open channel actuator 60. Outward movement of open channel actuator 60 causes the distal surface of open channel actuator base portion 62 to contact actuator engagement projection 48 thereby rotating jaw 40 to an open position. Similarly, when jaw 40 is in an open position, pulling inward or retraction of actuator control means 30 causes inward movement of open channel actuator 60. Inward movement of open channel actuator 60 causes the proximal surface of open channel actuator tip 66 to contact actuator engagement projection 48 thereby rotating jaw 40 to a closed position.

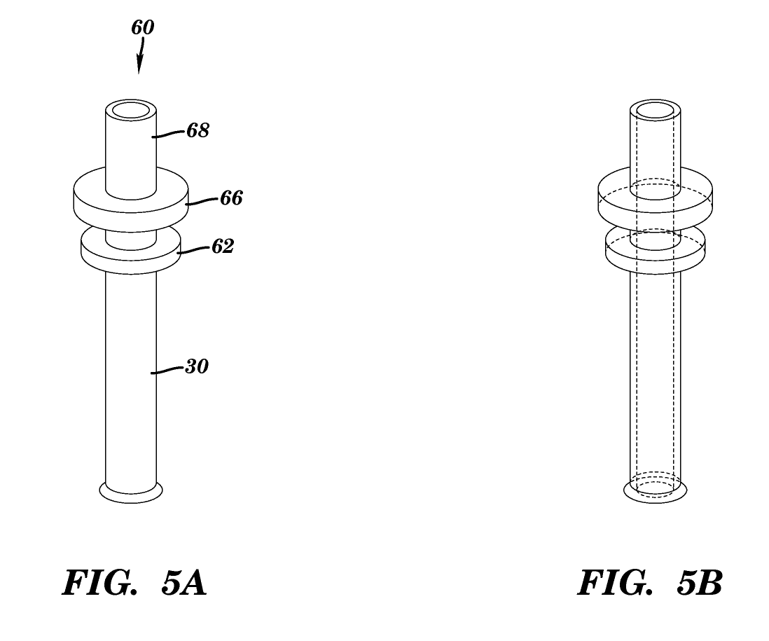

In another configuration for actuator 60, as shown in FIG. 5, wherein tip 66 is a substantially flat disk extended by tip extension 68.

An important feature of open channel actuator 60 and open channel actuator control means 30 is that they define an open channel or lumen through which various materials, tools, or accessories may freely pass.

An alternate embodiment of the invention may have a rigid, semi-rigid, or articulated shaft. Other embodiments may have a malleable shaft, allowing the user to form the shaft into a desired shape prior to insertion into the body. In malleable embodiments, the channel within the sheath 20 which houses the open channel actuator control 30 must be of sufficient size to allow the sheath 20 to be in a bent configuration and have sufficient room for the open channel actuator control 30 to also be bent and still to move freely in the longitudinal direction.

The parts of the biopsy forceps 10 may be created by any conventional method including, but not limited to, conventional machining, turning, boring, grinding, electrical discharge machining, casting, molding such as injection, thermoform, etc. or combinations thereof. The forceps can be fabricated in a wide size range for use in micro-surgery to conventional surgery. Current standard diameters include a wide range of instrument diameters between 1.0 and 10.0 mm. Both larger and smaller sizes may be created depending on the need of the user.

Many features have been listed with particular configurations, options, and embodiments. Any one or more of the features described may be added to or combined with any of the other embodiments or other standard devices to create alternate combinations and embodiments.

This invention describes designs for low cost biopsy forceps for use in endoscopes or other similar medical instruments that have working channels. The forceps were designed to meet the following key constraints: maximized central hollow cavity (channel or lumen) to allow additional functionality, maximum force applied in the closed position, and low cost assembly and manufacturability for a single-patient disposable. The central hollow cavity enables the forceps to be used with a central optical fiber or fiber bundle for concurrent optical measurements, such as elastic scattering spectroscopy, fluorescence spectroscopy, Raman spectroscopy, fluorescent microscopy, confocal microscopy, etc. The hollow channel is large enough to allow further functionalities, including additional tooling such as a water port for cleaning, snare for cauterization, spike for collection of multiple biopsy samples or stabilization, vacuum, etc; while maintaining enough volume in the forceps cavity to obtain sufficient tissue sampling for subsequent pathological analysis. The designs apply the maximum force in the closed position to allow sufficient gripping force to avulse/cut tissue for biopsy. By designing the kinematics in this way, we enable modifications of the design that reduce the constraints on the sharpness of the forceps jaws and thus enable alternative cheaper materials (e.g. plastic rather than metal). Simple and inexpensive design allows for the device to be a single-use or disposable item.

The key features of the designs include outside hinges or flexure and a ferrule that actuates the opening and closing of the forceps through either a cam geometry or through use of shape memory alloys. The simple and elegant designs have few parts for low cost assembly/manufacture and can be actuated in a number of ways. The preferred embodiment of the actuation is through a single wire. The design addresses a growing need for multifunctional tools in biopsy by enabling a standard forceps utility integrated with additional tooling. Moreover, the design improves on existing standard forceps designs by maximizing the force during closure which enables both better gripping action for the same applied force and use of lower cost materials such as plastic. Because the designs were conceived of with the constraints of low cost manufacturability, they have limited numbers of individual parts that can be easily and inexpensively assembled. By including additional functionality to the forceps, the user can be more efficient in the operation.

A variety of diagnostic and therapeutic tools can be integrated into the forcep. Optical fiber probes, electrical (e.g. impedance probes), cauterizing, cutting, injecting, grasping and hemostatic tools can be readily integrated into the forcep. Applications for such integrated tools include spectroscopic, microscopic, and/or sensor-guided biopsy/resection at flexible/rigid endoscopy. Virtually any hollow viscus or tissue space can be biopsied including: (1) the gastrointestinal and hepatobiliary tracts (via esophagogastroduodenoscopy, enteroscopy, endoscopic retrograde cholangiopancreatography, pancreatobiliary ductoscopy, colonoscopy, endoscopic ultrasonography); (2) the genitourinary tract (via cystoscopy, ureteroscopy), (3) the airways (via bronchoscopy); (4) the oral cavity and oropharyngeal structures; (5) the mediastinum (via mediastinoscopy), (6) the peritoneum (via laparoscopy and/or NOTES), (7) the joint spaces (via arthroscopy); (8) the cervico-vaginal region (via colposcopy); and (9) the cranium (via trans-sphenoidal approaches). Applications in laparoscopic surgery as well as natural orifice transluminal endoscopic surgery (NOTES) are envisioned as well.

Examples of known biopsy forceps include U.S. Pat. No. 6,129,683, U.S. Patent Publication No. 2008/0009857, U.S. Patent Publication No. 2007/0073185, U.S. Patent Publication No. 2006/0259070, U.S. Patent Publication No. 2006/0178699, U.S. Patent Publication No. 2005/0261735, U.S. Patent Publication No. 2005/0235735, U.S. Patent Publication No. 2004/0181169,U.S. Patent Publication No. 2003/00195432, U.S. Patent Publication No. 2003/0073928, U.S. Patent Publication No. 2002/0188220, U.S. Pat. No. 6,394,964, U.S. Pat. No. 6,174,291, U.S. Pat. No. 6,159,162, and U.S. Pat. No. 6,066,102, which are hereby incorporated by reference in their entirety. The various embodiments and examples of the present forceps differ from forceps in the background art by the features mentioned herein including allowing for fewer parts, lower cost manufacturing, maximized open central channel space, maximal force applied in the closed position, and ease of assembly.

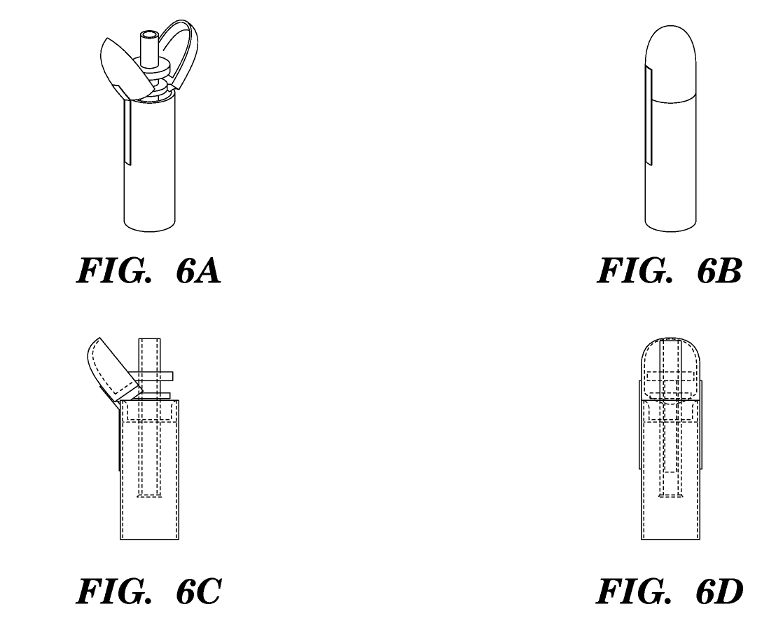

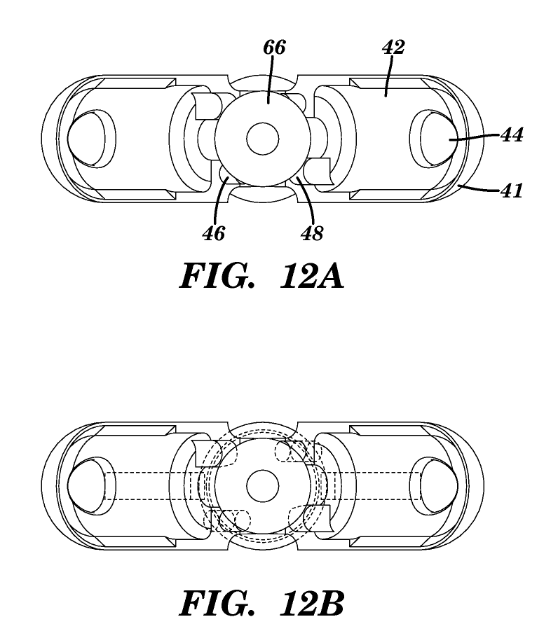

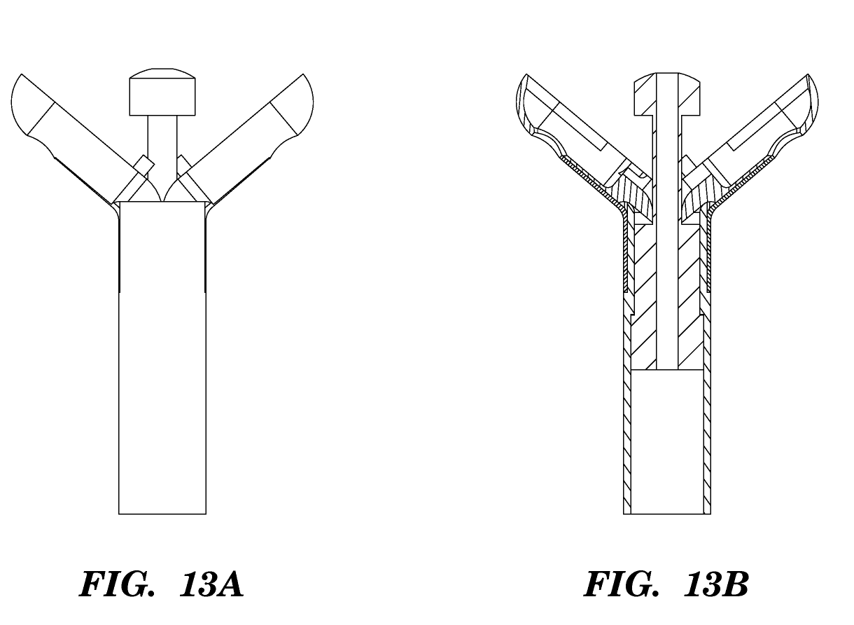

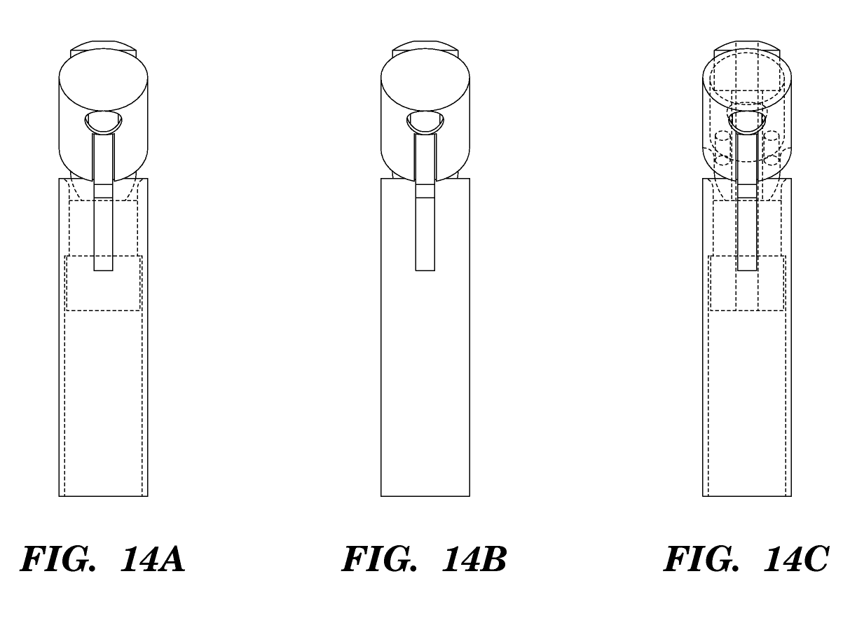

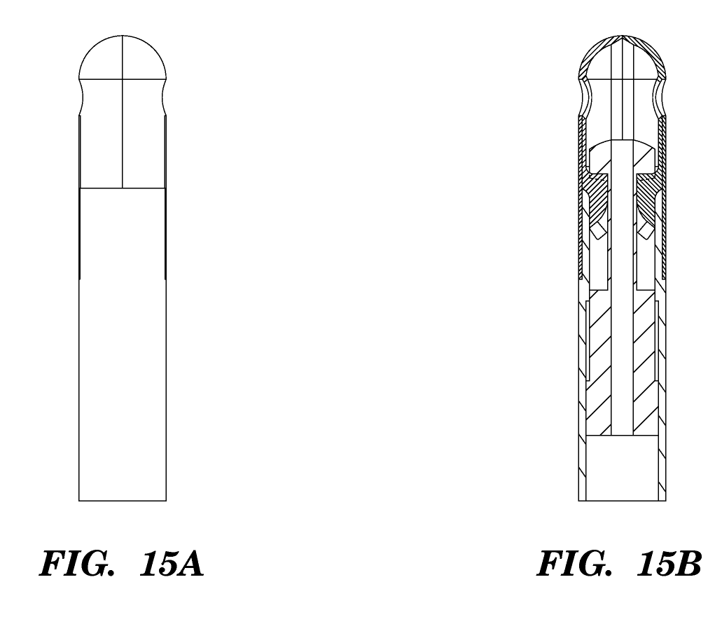

Embodiments of the present invention provides for a simplified biopsy forceps. This involves the use of fewer parts and/or fewer connections or linkages than in prior art systems which result in a forceps that is easier and less expensive to produce. Other forceps require complex hinging and linkages to control wires for operation of the forceps jaw or jaws. For example, see U.S. Patent Publication No. 2008/0009857 to Yanuma, U.S. Patent Publication No. 2007/0073185 to Nakeo, U.S. Patent Publication No. 2005/0261735 to Shibata, U.S. Patent Publication No. 2003/00195432 to Kortenbach, U.S. Pat. No. 6,394,964 to Sievert, U.S. Pat. No. 6,129,683 to Sutton, and U.S. Pat. No. 6,066,102 to Townsend. The greater number of connections or linkages contributes to increased play or distortion in movement of the jaws of the forceps. The greater number of connections or linkages also contributes to higher costs due to a greater number of parts and the associated costs of manufacturing and assembling a more complex device. Embodiments of the present invention utilize fewer parts such as a flexible hinging means 70 as described herein in combination with the actuator 60 as described herein (see, e.g., FIGS. 4, 7 and 12-15). In certain embodiments, an additional advantage of the flexible hinging arises from the substantially flush configuration of the hinging means with the outer surface of the housing or forceps jaw. Embodiments of the substantially flush configuration can be seen by way of examples in FIGS. 4 and 6. Embodiments of the present invention have the advantage of not requiring additional space around the housing for operation of the hinging means.

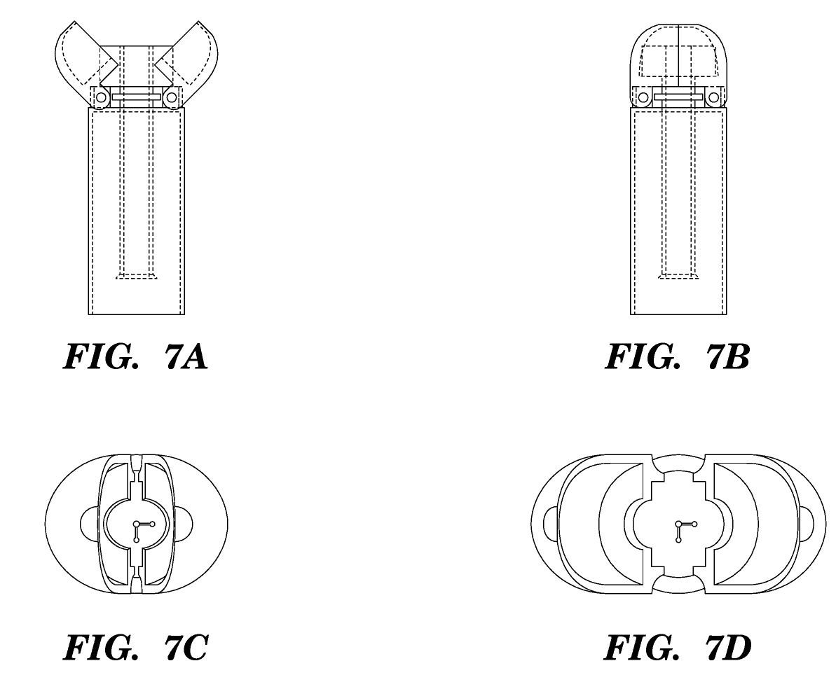

Embodiments of the present invention allow for a maximized open central channel. The design of such an open channel allows for further functionalities without sacrificing force during closure. Other forceps designs do not provide for an open control channel (for example, U.S. Patent Publication No. 2006/0259070 to Livneh, U.S. Patent Publication No. 2004/0181169 to Diamond, or U.S. Patent Publication No. 2002/0188220 to Kryzanowski) or do not allow for a maximized control channel due to the presence of control wires or other features (for example, U.S. Pat. No. 6,129,683 to Sutton). Moreover, as can be seen in FIGS. 7A and 7B, the present forceps feature outside hinges or flexures for ease of opening and closing.

This system results in improved endoscopic screening and surveillance by providing “real-time detection” data to guide biopsies and treatment by mucosal resection/ablation. This system enhances both disease detection and therapeutic ablation, and can thus assist prevention of deaths.

Noninvasive optical tissue diagnosis, often called “optical biopsy,” utilizing optical spectroscopy, is typically mediated by optical fibers, and has become a major component of the growing field of biomedical optics. These optical fibers are typically connected to monitoring means including as computers, etc. The most common approach has involved UV-light-induced fluorescence spectroscopy, although Raman spectroscopy and diffuse reflectance spectroscopy have also been investigated. ESS is a point spectroscopic measurement technique that, when performed using an appropriate fiberoptic geometry, is sensitive to morphological changes at the cellular and sub-cellular scale. These include nuclear size and chromaticity, chromatin granularity, nuclear crowding, and changes in the size/density of mitochondria and organelles. Clinical translational studies have been reported on the efficacy of ESS-based optical biopsy for distinguishing a range of diseases in various organ sites. ESS is a site-specific measurement—not an imaging modality—that samples a tissue volume of ≦0.05 mm3. The probe is in optical contact with the tissue under examination and has separate illuminating and collecting fibers. Each measurement takes about 30 msec, and it is possible to perform several measurements per second, limited by the time to move the probe from spot to spot. Surveillance of large mucosal areas are achievable using a rapid succession of point measurements while moving/scanning the probe over the mucosal surface.

As an example of the utility of an integrated optical biopsy system, consider the case of Barrett's esophagus (BE). The business of BE surveillance has a major efficiency barrier: At present, it is virtually impossible to distinguish dysplastic Barrett's esophagus (DBE) from non-dysplastic Barrett's esophaguse (NDBE) endoscopically. Visually, BE appears as an apparent proximal displacement of the squamocolumnar junction often with associated proximal mucosal tongues/islands. Diagnosis can be established post facto by histopathological interpretation of forcep biopsies. When BE is detected, patients are subsequently surveyed every 1-2 years to monitor for disease progression and dysplasia, or pre-cancer. Surveillance biopsies in BE are obtained randomly using a geometric pattern known as the “Seattle Protocol” whereby 4-quadrant biopsies are obtained every 2 cm within the BE segment. For example, in a 6 cm BE segment, four biopsies at three distinct levels (i.e. 12 physical biopsies) are required. On average, 5-6 biopsies are obtained per BE surveillance endoscopy. While, case-control studies have shown only modest utility for BE surveillance in the early detection and prevention of EAC1, a subset of patients will advance along the dysplasia-carcinoma sequence to high-grade dysplasia (HGD). HGD is considered imminently cancerous and surgical resection, ablation, or endoscopic mucosal resection (EMR) is recommended. It is generally accepted that detection at the earliest stages of dysplasia results in better outcomes. As such, given the poor utility of random biopsies as a screening/surveillance method and the need for early detection of dysplasia in BE, there is a clear need for tools that enhance the targeting of biopsies for the detection of dysplasia.

Guided biopsy tools represent a major step forward in dysplasia detection in the GI tract as well as in other organ systems. Beyond surveillance of Barrett's esophagus, major applications include detection, surveillance, biopsy and ablation of colonic and gastric polyps, flat dysplasia in chronic inflammatory bowel disease, and cervical and bladder dysplasia. Here, we present a design for a jaw-type biopsy forceps that is modified to allow a hollow central channel through which a fiberoptic probe can be integrated. (Note: other functionalities can also be included as discussed herein). The open jaw is placed in apposition to the mucosa and the fiber probe makes contact with the mucosa. After measurements are obtained, if the optical measurement indicates the desired target, e.g., suspected dysplasia, the jaws would be closed, and the mucosa avulsed, obtaining the biopsy in the usual manner. Cautery ablation is possible as well. The present enhanced but user-friendly familiar tool can readily be adopted into current practice and, as such, has large commercial potential. Not only with biopsy/histopathology as the primary diagnostic method, but rather to use optical measurements to provide real-time guidance for selective biopsy, with the goal of significantly reducing the number of unnecessary biopsies (increased specificity), while, nonetheless, increasing the yield (sensitivity). The additional goal is to properly guide ablation of dysplastic tissue once it is detected. The result is faster procedures for both detection and treatment, and an overall reduction in the cost of health care. We believe that such tools would be readily accepted into practice with commensurate rapid commercial potential.

One tool currently on the market, manufactured by SpectraScience, consists of an integrated forceps with a single optical fiber through the center. However, this design is limited by the available space in the hollow chamber as it is partially occluded by the jaw and actuation mechanisms, is made of many parts and is thus expensive, and the design has its force maximum in the open position limiting the jaw material choice. In contrast, our designs provide a significantly larger central channel diameter to enable incorporation of multiple functionalities (e.g. fiber bundles, fiber and water jet, fiber and vacuum, etc.), apply the maximum force the forceps are closed enabling low cost materials choices, and is made of few parts for low-cost assembly during manufacture.

The concepts described allow for a center hollow channel in biopsy forceps. The center channel can be used as a working channel for wide range of tools such as optical sensors, water flushing, spike, syringe, multiple tissue collections, suction, snares, cauterizing functionality, etc. For example when used with an optical sensor, the forceps become both a biopsy and diagnostic tool. Furthermore, the actuation concepts described, can be used with or without the center channel.

In some embodiments, the present invention may be defined in any of the following numbered paragraphs:

1. An endoscopic biopsy forceps with an open central channel, comprising: a sheath having a proximal end and a distal end; a housing connected with said distal end of said outer sheath; an open channel actuator control means having a proximal end and a distal end and passing through said sheath; an operating means attached to said proximal end of said open channel actuator means; an open channel actuator attached to a distal end of said open channel actuator control means and having a first projection and second projection; a first jaw having an actuator engagement projection, said first jaw having a first position and a second position; and a second jaw having an actuator engagement projection, said second jaw having a first position and a second position, wherein said first jaw and said second jaw are movably connected to said housing, and wherein when said open channel actuator is moved longitudinally along a body of the instrument, said first jaw moves between the open position and the closed position.
2. The endoscopic instrument of paragraph 1 further comprising a hole extending through at least one of said first jaw and said second jaw.
3. The endoscopic instrument of paragraph 1 or 2 wherein said jaws are pivotally connected to said housing by a flexible hinging means.
4. The endoscopic instrument of any of paragraphs 1-3 wherein said jaws have a cutting edge extending along an upper portion of said jaws.

### EXAMPLES

### Example 1

### Side Hinge Concept

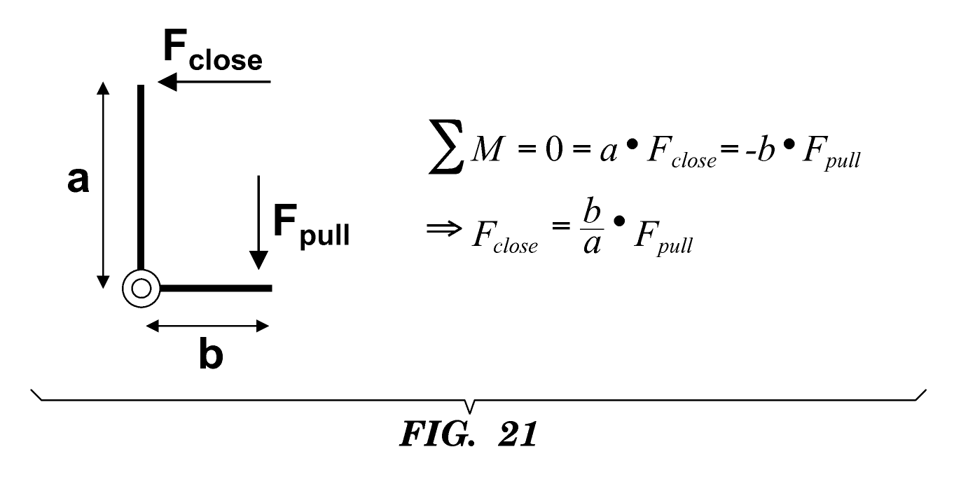

The hinges are offset from the center axis on the side of the tube, rather than in the middle as is done on conventional designs. This allows for maximal use of the internal space for additional functionalities. Furthermore, this design has the advantage of increased closing force as compared to the conventional mid-tube hinge design. See FIG. 21.

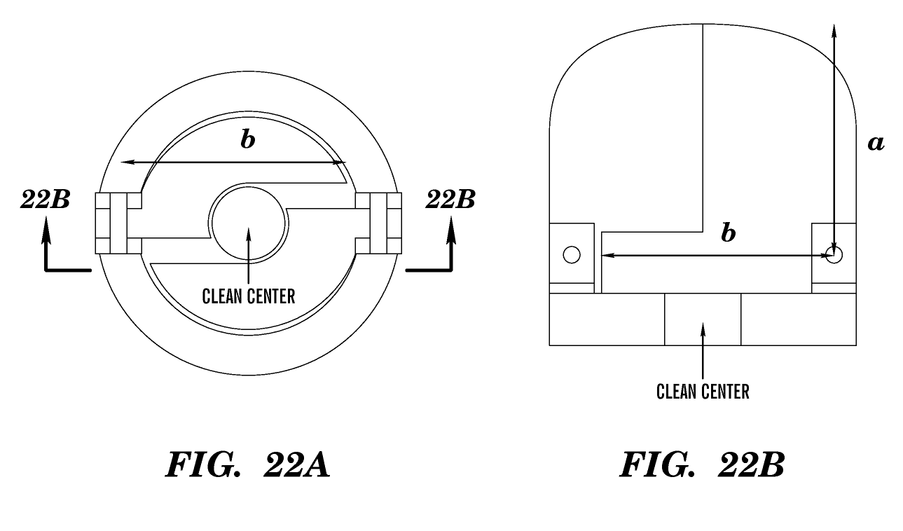

The length of the jaws “a” can be variable depending on the desired forces needed, and the length of “b” is limited by the tube size (See FIG. 22) in that the forceps can fit through the working channel of the medical scope. Here, the hinge sees an additional moment due to the twisting caused by asymmetric jaws. The hinge itself could be a pin hinge, a screw hinge with one or two screws, rivet, or any other equivalent attachment technique. The materials used for the hinge and jaws could be metal, plastic, or ceramic.

### Example 2

### Ferrule Design

The jaws are opened and closed by using the CAM ferrule geometry (see FIGS. 7A-D). The ferrule is a rigid tube that contains the fiber-optics, and/or other components to provide additional functionalities to the forceps (see FIG. 5). The ferrule has physical features on the outside of the tube that mechanically open and close the forceps jaws. These features for example, could be rings or tabs which are a part of the ferrule or which are attached to the ferrule. The shape of the contact geometry (areas where ferrule comes in contact with the jaws) controls the opening and closing. The ferrule can be metal, plastic, and/or ceramic.

### Example 3

### Flex Joint Concept This concept uses a flexible hinge to constrain the jaws (see FIG. 6). At least one flexible strip is needed for each jaw. Alternatively, the flexible hinge may be a sleeve. The flexible hinge extends from the tube to the jaw. The hinge can be attached between the tube and the jaw in a number of ways. For example, the strip can be a slot fit, welded, soldered, glued, melted, a single piece with the jaws or tube attached to the other member, etc. The strip material can be metal, plastic, textile, or made of any other flexible material. The jaws are actuated by the ferrule as described above. With this design, longer jaw arm geometry is possible which creates a higher closing force.

### Example 4

### Shape Memory Concept

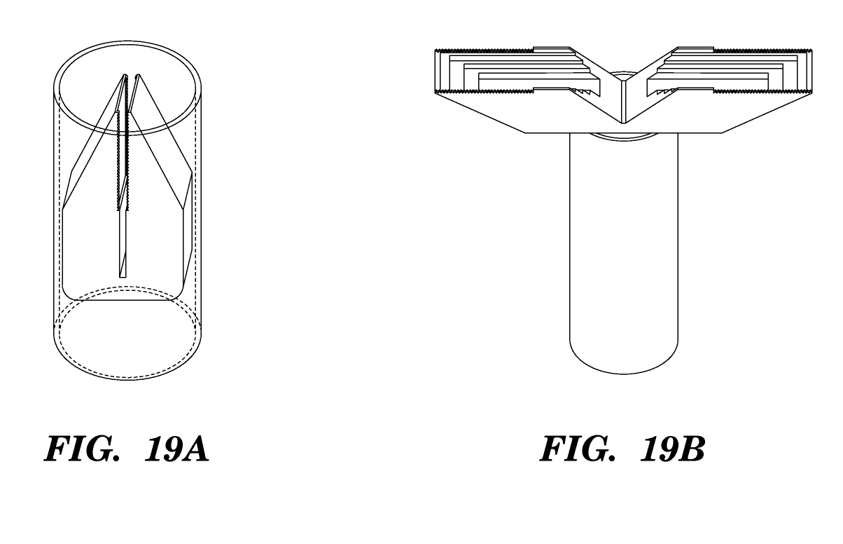

This concept utilizes the metal shape memory properties to open the jaws. The closing action of the jaws comes from the downward pulling action on the jaws against the tube ledge to collapse the jaws (see FIG. 19).

### Example 5

### Actuation Mechanism

The jaws in each of the above mentioned concepts can be actuated in a number of ways. One such way is by wire. The wire may be metal, plastic, or from another material. The wire is attached to the ferrule, and extends through the length of the tube to the user. The user then manipulates the wire directly or indirectly to create the translational movement. A variation of this can be a wire-spring combination. A preloaded spring is mounted between the ferrule and a plate attached to the wire. As the wire is actuated the spring delivers force to actuate the jaws. The spring can be produced in a number of ways including geometry, a flexible hinge, etc.

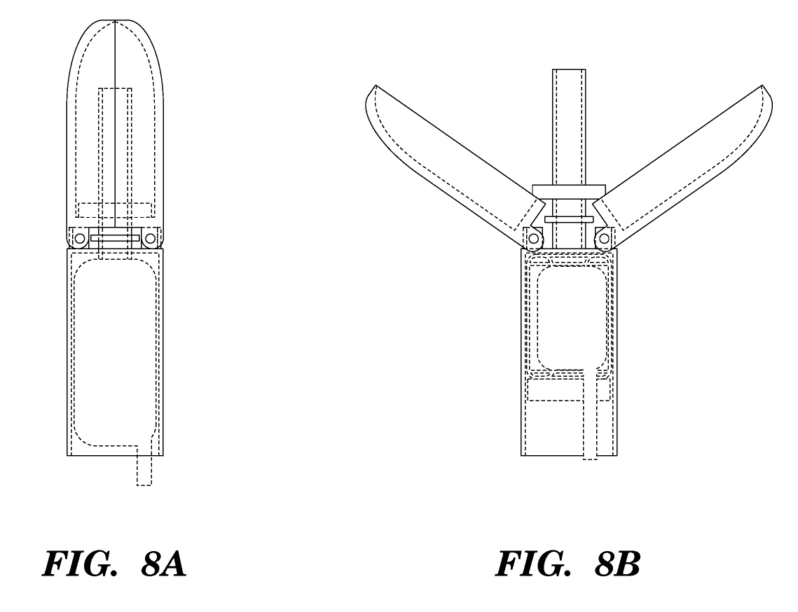

Another actuation mechanism is a pressure actuation (see FIG. 8). With this method, there is a space between the bottom of the jaws and a plate in the tube. This space, for example, can be hermetically sealed, or a deflated balloon, that when filled with gas or liquid a change of volume will occur resulting in a translational movement of the ferrule.

### Example 6

### Additional Functionality with Respect to ESS

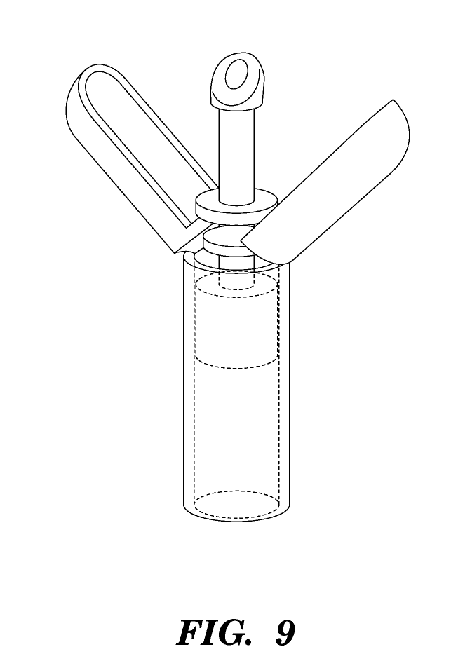

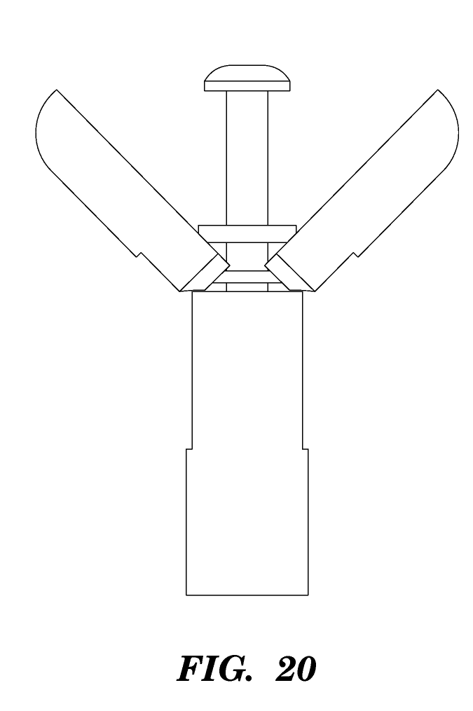

The additional enclosed volume in the jaws provided in these designs as compared to existing designs allows for two specialized modalities in combination with elastic scattering spectroscopy. First, one can incorporate an angled probe (e.g. 45 degrees) which provide enhanced performance (see FIG. 9). Second, the tip of the dual fiber can be enclosed in an expanded ferrule head that resembles a mushroom cap (see FIG. 20) to allow for dragging across the mucosa without tearing or puncturing the tissue. This design enables rapidly, real-time measurements of a much larger area as opposed to point measurements.

Although the examples given include many specificities, they are intended as illustrative of only one possible embodiment of the invention. Other embodiments and modifications will, no doubt, occur to those skilled in the art. Thus, the examples given should only be interpreted as illustrations of some of the preferred embodiments of the invention, and the full scope of the invention should be determined by the appended claims and their legal equivalents.

1. An endoscopic instrument comprising:
an outer sheath having a proximal end and a distal end;
a housing connected with the distal end of the outer sheath;
an actuator control having a proximal end and a distal end that can pass through said sheath;
wherein an actuator is attached to the distal end of the actuator control and having a first projection and second projection;
an operator attached to said proximal end of the actuator;
a first jaw having an actuator engagement projection, said first jaw having a first closed position and a second open position; and
a second jaw having an actuator engagement projection, the second jaw having a first closed position and a second open position,
wherein said first jaw and the second jaw are movably connected to the housing, and wherein when the actuator is moved longitudinally along the sheath, the first jaw moves between the closed position and the open position.

2. The endoscopic instrument of claim 1 further comprising a hollow central channel in the actuator and actuator control through which an object can be inserted.

3. The endoscopic instrument of claim 1, wherein the first and the second jaw is pivotally connected to the housing by a flexible hinge.

4. The endoscopic instrument of claim 1 wherein the first and the second jaw has a cutting edge extending along at least one portion of the jaws.

5. The endoscopic instrument of claim 1, wherein said flexible hinge and jaw comprises a metal, metal alloy, or shape memory alloy.

6. The endoscopic instrument of claim 1, wherein the jaws are cup-shaped.

7. The endoscopic instrument of claim 6, wherein one or both the first and the second jaw includes a hole to allow fluid or material to escarp as the first and the second jaw close.

8. The endoscope instrument of claim 1, wherein the first and the second jaws is a grasper.

9. The endoscopic instrument of claim 1, wherein the actuator control is an open-channel multi-strand cable.

10. The endoscopic instrument of claim 1, wherein the outer sheath is selected from the group consisting of flexible polymeric tubing and coiled steel.

11. The endoscopic instrument of claim 2, wherein an object is inserted in the hollow central channel, and wherein the object comprises an optical fiber or fiber bundle for optical measurements.

12. The endoscopic instrument of claim 11, wherein the optical measurements are made by being connected to an instrument selected from the group consisting of elastic scattering spectroscopy, fluorescent spectroscopy, diffuse reflection spectroscopy, Raman spectroscopy, fluorescent microscopy, confocal microscopy, and combinations thereof.

13. The endoscopic instrument of claim 2, wherein the object included in said hollow central channel comprises a water port, a snare, a spike, a vacuum line, or a cauterization tool.

14-17. (canceled)

18. A method of obtaining a target sample from a subject comprising
(a) inserting the instrument of claim 1 in a subject;
(b) taking optical measurements of tissue;
(c) moving the instrument from place to place; and
(d) evaluating the optical measurements of step (b).

19. The method of claim 18, further comprising ablating a tissue sample of the reading from step (b) meet a specific criteria.

20. The method of claim 18, further comprising disposing of the instrument after use.
